(19) United States
(12) Patent Application Publication
(10) Pub. No.: US 2011/0023158 A1
(43) Pub. Date: Jan. 27, 2011

(54) BOVINE GENOME EDITING WITH ZINC FINGER NUCLEASES

(75) Inventors: Joseph Bedell, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,188

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; C12N 5/071

(52) U.S. Cl. 800/15; 435/325

(57) ABSTRACT

The present invention provides a genetically modified bovine or cell comprising at least one edited chromosomal sequence. In particular, the chromosomal sequence is edited using a zinc finger nuclease-mediated editing process. The disclosure also provides zinc finger nucleases that target specific chromosomal sequences in the bovine genome.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified bovine or bovine cells comprising at least one edited chromosomal sequence. In particular, the invention relates to the use of targeted zinc finger nucleases to edit chromosomal sequences in the bovine.

### BACKGROUND OF THE INVENTION

The cattle industry is a vital economic component of our economy, which produces many essential and varied products including dairy products (e.g. milk, cheeses, creams, yogurt, butter and more), meat, and concentrated protein products derived from beef.

Many phenotypic traits associated with milk and meat production have been identified. The genetics of these phenotypes are well documented, but in some cases the actual genes that are responsible are yet to be characterized. The identification of genes controlling several traits of interest in cattle has been accomplished by positional candidate cloning. Once the location of a trait is determined by linkage to the markers, possible candidate genes controlling the trait can be inferred because of their proximity to linked markers. Subsets of genes that are mapped in humans and mice have also been mapped in cattle through comparative genomic study. Bovine genetic map was published and updated by the National Center for Biotechnology Information (NCBI) and is available at http://www.ncbi.nlm.nih.gov/projects/genome/guide/cow/. Other informational databases on the genetic maps of cattle have also been done.

In addition to milk and meat production, traits such as disease resistance, coat color and breeding are also important for the cattle industry. There is a need, therefore, for improved methods of knocking out genes coding undesirable proteins in cattle, as well as means of modifying genes involved in desirable phenotypes for higher economic value.

### SUMMARY OF THE INVENTION

One aspect of the present disclosure encompasses a genetically modified bovine comprising at least one edited chromosomal sequence.

A further aspect provides a bovine embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

Another aspect provides a genetically modified bovine cell comprising at least one edited chromosomal sequence.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with bovine- or human-related diseases or bovine traits. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with bovine- or human-related diseases or bovine traits generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with bovine- or human-related diseases or bovine traits using targeted zinc finger nuclease technology is rapid, precise, and highly efficient.

### (I) Genetically Modified Bovine

One aspect of the present disclosure provides a genetically modified bovine in which at least one chromosomal sequence encoding a disease- or trait-related protein has been edited. For example, the edited chromosomal sequence may be inactivated such that the sequence is not transcribed and/or a functional disease- or trait-related protein is not produced. Alternatively, the edited chromosomal sequence may be modified such that it codes for an altered disease- or trait-related protein. For example, the chromosomal sequence may be modified such that at least one nucleotide is changed and the expressed disease- or trait-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed disease- or trait-related protein comprises a single amino acid deletion or insertion, provided such a protein is functional. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. The modified protein may have altered substrate specificity, altered enzyme activity, altered kinetic rates, and so forth. Furthermore, the edited chromosomal sequence may comprise an integrated sequence and/or a sequence encoding an orthologous protein associated with a disease or a trait. The genetically modified bovine disclosed herein may be heterozygous for the edited chromosomal sequence encoding a protein associated with a disease or a trait. Alternatively, the genetically modified bovine may be homozygous for the edited chromosomal sequence encoding a protein associated with a disease or a trait.

In one embodiment, the genetically modified bovine may comprise at least one inactivated chromosomal sequence encoding a disease- or trait-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional disease- or trait-related protein is not produced. The inactivated chromosomal sequence comprises no exogenously introduced sequence. Such a bovine may be termed a “knockout.” Also included herein are genetically modified bovines in which two, three, four, five, six, seven, eight, nine, or ten or more chromosomal sequences encoding proteins associated with a disease or a trait are inactivated.

In yet another embodiment, the genetically modified bovine may comprise at least one chromosomally integrated sequence. The chromosomally integrated sequence may encode an orthologous disease- or trait-related protein, an endogenous disease- or trait-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a bovine or human disease- or trait-related protein may be integrated into a “safe harbor” locus, such as the Rosa26 locus, HPRT locus, or AAV locus. In one iteration of the disclosure, an animal comprising a chromosomally integrated sequence encoding disease- or trait-related protein may be called a “knock-in”, and it should be understood that in such an iteration of the animal, no selectable marker is generally present. The present disclosure also encompasses genetically modified animals in which two, three, four, five, six, seven, eight, nine, or ten or more sequences encoding protein(s) associated with a disease or a trait are integrated into the genome.

In an exemplary embodiment, the genetically modified bovine may be a “humanized” bovine comprising at least one chromosomally integrated sequence encoding a functional human disease or trait-related protein. The functional human disease or trait-related protein may have no corresponding ortholog in the genetically modified bovine. Alternatively, the wild-type bovine from which the genetically modified bovine is derived may comprise an ortholog corresponding to the functional human disease or trait-related protein. In this case, the orthologous sequence in the “humanized” bovine is inactivated such that no functional protein is made and the “humanized” bovine comprises at least one chromosomally integrated sequence encoding the human disease or trait-related protein. Those of skill in the art appreciate that “humanized” bovines may be generated by crossing a knock out bovine with a knock in bovine comprising the chromosomally integrated sequence.

The chromosomally integrated sequence encoding a disease or trait-related protein may encode the wild type form of the protein. Alternatively, the chromosomally integrated sequence encoding a disease- or trait-related protein may comprise at least one modification such that an altered version of the protein is produced. In some embodiments, the chromosomally integrated sequence encoding a disease or trait-related protein comprises at least one modification such that the altered version of the protein produced causes a disease or forms a trait. In other embodiments, the chromosomally integrated sequence encoding a disease- or trait-related protein comprises at least one modification such that the altered version of the protein protects against the development of a disease or an undesirable trait.

In yet another embodiment, the genetically modified bovine may comprise at least one edited chromosomal sequence encoding a disease or trait-related protein such that the expression pattern of the protein is altered. For example, regulatory regions controlling the expression of the protein, such as a promoter or transcription binding site, may be altered such that the disease or trait-related protein is over-produced, or the tissue-specific or temporal expression of the protein is altered, or a combination thereof. Alternatively, the expression pattern of the disease or trait-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a disease or trait-related protein. The genetically modified bovine comprising the lox-flanked chromosomal sequence encoding a disease or trait-related protein may then be crossed with another genetically modified bovine expressing Cre recombinase. Progeny comprising the lox-flanked chromosomal sequence and the Cre recombinase are then produced, and the lox-flanked chromosomal sequence encoding a disease or trait-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a disease or trait-related protein.

Exemplary examples of bovine chromosomal sequences to be edited include those that code for proteins related to milk production, quality and processing, such as caseins, lactose and lactose-related proteins (e.g. galactosidase, lactase, galactose, beta lactaglobulin, alpha lactalbumin, lactoferrin), osteopontin, acetyl coA carboxylase, tyrosinases and related proteins, regeneration inducing peptide for tissues and cells (RIPTAC) and other growth hormones. Other exemplary examples include those proteins related to meat production and quality such as FGFR3 and EVC2 and MC1R. Those of skill in the art appreciate that other proteins are involved in BSE-resistance, coat color and quality, environmental impact, and breeding, but the genetic loci have not necessarily been determined.

Six proteins produced by the cow's mammary gland during lactation represent over 90% of the total protein produced in milk. These are the four caseins (as1, as2, β and k) and the two major whey proteins, β-lactoglobulin and a-lactalbumin.

TABLE 1
Approximate average milk composition of cattle

| | Concentration or | |
|---|---|---|
| | percentage in milk | |
| Milk component | Production level | |
| Fat | 3.7% | |
| Lactose | 4.6% | |
| Protein | 3.1% | |
| Total Casein | 27.3 | g/l |
| as1-Casein | 10 | g/l |
| as2-Casein | 3.4 | g/l |
| β-Casein | 10 | g/l |
| k-Casein | 3.9 | g/l |
| a-Lactalbumin | 1.2 | g/l |
| β-Lactoglobulin | 3.0 | g/l |

The caseins combine with calcium to form micellar structures that remain soluble in milk and are of functional importance in cheese making. Caseins form the curd in cheese and the amount of casein present in milk determines the cheese yield of the milk. The caseins have an open structure and are hydrated despite the high content of hydrophobic amino acids present in the molecule. These hydrophobic surfaces on the molecule are important in casein-casein interactions that are partially responsible for the high viscosity of casein solutions and for its foaming and emulsification properties.

Bovine β-Casein is 209 amino acids in length and is rich in the amino acid proline. It contains 5 phosphorylated serines clustered in the first 35 amino acids at the N-terminal end of the molecule and the rest of the protein is very hydrophobic. This hydrophobic nature of β-casein has been correlated with excellent emulsifying, foaming and gelling characteristics in manufacturing processes.

One modification that can be made to β-casein is the deletion of a plasmin cleavage site. Plasmin is one of the major milk proteases that is found at varying levels in the milk of all cows. Making this alteration to the β-casein molecule prevents plasmin from cutting it to form two smaller molecules that have different properties than intact β-casein. One of the peptides produced by this cleavage causes a bitter flavor in cheese. The modifications made to the β-casein molecule will prevent this cleavage and prevent the formation of these bitter peptides in cheese as well as in milk.

The second alteration of β-casein that can be made is the removal of the cleavage site for chymosin. Chymosin is the enzyme used in cheese-making to start the formation of the curd. It cleaves a portion of the k-casein molecule exposing the inner contents of the casein micelle to the solution, thus causing precipitation of the a and β-caseins and the formation of the cheese curd.

However, a third modification would be adding chymosin (protease) cleavage sites, which would increase the rate of cheese ripening.

A fourth modification that can be made to β-casein is the addition of glycosylation sites to the molecule. A glycosylation site causes a carbohydrate to be attached to the casein molecule. The addition of a carbohydrate to a protein increases its hydrophilicity. The formation of a glycosylated β-casein in milk should increase the solubility of β-casein and modify other functional properties such as viscosity, water holding capacity, foaming and emulsification. Proof for an increase in solubility of β-casein after glycosylation has been reported by a number of groups, but the behavior of this casein in milk has yet to be analyzed. The addition of a carbohydrate to the casein molecule will also affect casein micelle structure and may reduce the size of the micelles. The reduction in casein micelle size has been shown to be beneficial in a number of manufacturing processes.

A fifth modification would be a modification of the single nucleotide polymorphism (SNP) that results in the one amino acid difference between the A1 and A2 beta casein types. The A1 and A2 beta caseins are the usual forms of beta casein found in dairy cow's milk. Cows can have only the A1 or only the A2 production trait but most dairy cows carry two different traits for beta casein production, so produce both A1 and A2 beta casein in their milk.

The A1 and A2 beta-caseins have since given rise to a number of rare minor subvariants. The same amino acid difference at position 67 occurs between minor variants, which on the basis of the amino acid present at position 67 may be classified as ‘A1 like’ or ‘A2 like’. Minor variants include A3, D and E, which like A2, contain a Proline at position 67; and B, C and F which, like A1, contain a Histidine and produce the same major digestion products as A1. To the extent that evidence exists that makes a particular variant or subvariant of beta caseins advantageous or disadvantageous, modifications may be made according to the present invention to produce the desired variant or subvariant.

Increased caseins in general (including alpha S1, alpha S2, beta and kappa) or an increased kappa to beta ratio has been shown to increase cheese yield. For example, fortification of skim milk with β-casein at a concentration 30% of that normally found in milk caused a 50% increase in curd firmness, indicating that the functional interactions of the caseins were increased possibly by additional electrostatic and hydrophobic interactions resulting from interactions of calcium and β-casein. The addition of the purified β-casein also causes an increase in the cheese yield of the milk.

Therefore, in one embodiment, the genetically modified bovine may comprise an edited chromosomal sequence encoding one or more proteins, or any combination thereof, including but not limited to alpha s1 casein, beta casein, kappa casein, alpha s2 casein, plasmin and/or chymosin wherein the edited chromosomal sequence comprises a mutation such that an improved cheese product can be made from the milk produced by the bovine. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. For example, the nonsense, deletion, or insertion mutation “inactivates” the sequence such that cleavage site for plasmin is not produced. Thus, a genetically modified bovine comprising an inactivated chromosomal sequence for the plasmin cleavage site may produce milk, and subsequently cheese, that is less bitter.

However, modifications or deletions may also be made that knock out the major caseins and other major milk proteins. As noted above, milk contains alpha s1 casein, beta casein, kappa casein, alpha s2 casein, beta lactoglobulin, alpha lactalbumin, and bovine serum albumin with relative abundance of 30:30:10:12:10:4:1. A milk allergy occurs when the immune system mistakenly sees the milk protein as something the body should fight off. This starts an allergic reaction, which can cause an infant to be fussy and irritable, and cause an upset stomach and other symptoms. Most children who are allergic to cow's milk also react to goat's milk and sheep's milk, and some of them are also allergic to the protein in soy milk, so viable alternatives are few. A person of any age can have a milk allergy, but it is more common among infants (about 2% to 3% of babies).

Knocking out these major cow proteins would produce a highly-purified, reduced allergy milk product. Reduced-protein milk that retains the fat content would be useful in feeding to infants and children with milk protein allergy as milk protein is the basis for most commercial baby formulas and goat, sheep and soy milk are often not effective alternatives.

Casein is the curd that forms when milk begins to sour, and whey is the watery part that is left after curd is removed. Beta and/or kappa caseins are responsible for curd allergy. Modification or deletion of beta and/or kappa caseins may reduce or eliminate curd allergy when milk is ingested.

The major whey proteins in cow's milk are beta-lactoglobulin and alpha-lactalbumin. Alpha-lactalbumin is an important protein in the synthesis of lactose and its presence is central to the process of milk synthesis. Other whey proteins are the immunoglobulins and serum albumin, enzymes, hormones, growth factors, nutrient transporters, disease resistance factors and others.

Because most whey proteins are not completely digested in the human intestine, the intact protein may stimulate a localized intestinal or systemic immune response known as whey allergy. Modification or deletion of one or more whey proteins may reduce or eliminate whey allergy when milk is ingested. Knocking out alpha-lactalbumin has also been shown to result in the production of a viscous, concentrated milk product. Thus, alpha-lactalbumin knock-out cows would likely produce a low-allergy concentrated milk, again very useful for infant formulas and other milk-based products for those with milk protein or whey allergy.

Another example of a milk protein that could be targeted for deletion is beta lactoglobulin (BLG) to decrease whey allergy. The function of BLG is not clear although it is similar in structure to retinol-binding protein and lipocalycins, suggesting that BLG may have a role in the transport of fatty acids and vitamin A. The ovine BLG gene promoter has been extensively characterized in both transgenic mice and mammary epithelial cells (MEC) in culture.

Therefore, in a further embodiment, the genetically modified bovine may comprise an edited chromosomal sequence encoding one or more proteins, or any combination thereof, including but not limited to alpha s1 casein, beta casein, kappa casein, alpha s2 casein, beta lactoglobulin, alpha lactalbumin, and/or bovine serum albumin, wherein the edited chromosomal sequence comprises a mutation such that an allergy is not produced. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that protein is not produced. Thus, a genetically modified bovine comprising an inactivated alpha s1 casein, beta casein, kappa casein, alpha s2 casein, beta lactoglobulin, alpha lactalbumin and/or bovine serum albumin chromosomal sequence may produce a low-allergenic milk product.

Modifications or deletions may be made that reduce or knock out the lipids present in milk such as the fatty acids, glycerides and sterols, resulting in defatted milk (or a “skim milk” cow). This would eliminate the need for “skimming” in the manufacturing process. For example, acetyl CoA carboxylase, which is a large and highly complex enzyme, regulates the rate of fat synthesis within the mammary gland. A modification or deletion in the amount of this enzyme would lead to a dramatic reduction in the fat content of milk and reduce the energy required by the animal to produce milk.

RIPTAC (Regeneration Inducing Peptide for Tissues and Cells) has recently been noted to support weight loss while retaining muscle mass. In one study administration of RIPTAC to mice at 2.5 micrograms/day increased muscle mass by 5% in two weeks. While the RIPTAC protein is of extremely low dosage in ordinary cow's milk, the ability to increase the production of this protein in cow's milk could produce a milk product capable of supporting exercise and weight loss while avoiding associated metabolic syndromes—in other words, a “slim milk.”

However, it has been shown that a higher-fat milk could be produced with a lysine to alanine substitution (K232A) in the diacylglycerol acyl transferase 1 (DGAT1) gene. This gene controls the last step of making milk fat, and the mutation makes it so the milk is higher in fat. Sometimes it is better for a farmer if the milk from their dairy cattle has a high percentage of fat, because milk fat is important in making butter and certain types of cheese.

Therefore, in another embodiment, the genetically modified bovine may comprise an edited chromosomal sequence encoding one or more proteins, or any combination thereof, including but not limited to acetyl CoA carboxylase, RIPTAC or diacylglycerol acyl wherein the edited chromosomal sequence comprises a mutation such that a higher or lower fat milk is produced by the bovine. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that the acetyl CoA carboxylase protein is not produced. Thus, a genetically modified bovine comprising an inactivated or modified acetyl CoA carboxylase chromosomal sequence may produce cows that make skim milk.

Lactose intolerance is the inability to metabolize lactose, because of a lack of the required enzyme lactase in the digestive system. It is estimated that 75% of adults worldwide show some decrease in lactase activity during adulthood. The frequency of decreased lactase activity ranges from as little as 5% in northern Europe, up to 71% for Sicily, to more than 90% in some African and Asian countries.

Reduction in lactose in milk may be accomplished through an insertion or modification that enables production of an enzyme such as lactase in milk. Lactase breaks up lactose into the two simple sugars glucose and galactose. Producing lactose-free milk at the animal level may allow for the production of lactose free dairy products without having to modify the milk in the dairy plant. Additionally, galactose-free milk could also be produced by through an insertion or modification that produces beta galactosidase. The sugars in milk could also be reduced by insertion of glucosidase to digest the glucose.

A second approach would be to insert a reversibly inactive lactase that, rather than being active in the milk production, as noted above, is active upon human digestion in the gastrointestinal tract.

Therefore, in another embodiment, the genetically modified bovine may comprise an edited chromosomal sequence encoding one or more proteins, or any combination thereof, including but not limited to lactose, lactase, galactose, beta galactosidase, glucose and glucosidase, wherein the edited chromosomal sequence comprises a mutation such that a lactose allergic reaction is not produced when the bovine milk is consumed by humans. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the insertion mutation here would produce a sequence for a protein such as lactase such that lactose protein is not produced in the harvested milk, but rather digested into galactose and glucose. Thus, a genetically modified bovine comprising an insertion of the chromosomal sequence for lactase may produce lactose-free milk.

Milk production may also be increased by an insertion or modification that would result in increased bovine somatotropin (BST) or bovine growth hormone (BGH) in bovine. Insufficient production of BST in cattle has also been associated with extreme dwarfism in cattle. Milk production could also be enhanced by a modification that would result in the increase of alpha lactalbumin production which results in increased milk production.

Finally, osteopontin (OPN) is a highly phosphorylated glycoprotein whose gene has been cloned and sequenced in different species. Several whole genome scans have identified quantitative trait loci (QTL) affecting milk production traits on bovine chromosome 6 close to the osteopontin gene (OPN) location. A single nucleotide polymorphism in intron 4 (C/T) was detected and primers were designed to amplify genomic DNA from 1362 bulls obtained from Cooperative Dairy DNA Repository and from 214 cows from the University of Wisconsin herd. The C allele was associated with an increase in milk protein percentage and milk fat percentage.

Therefore, in yet a further embodiment, the genetically modified bovine may comprise an edited chromosomal sequence encoding one or more proteins, or any combination thereof, including but not limited to alpha lactalbumin, BST, BGH, and OPN wherein the edited chromosomal sequence comprises a mutation such that milk production in bovine increases when compared with bovine with an unedited chromosomal sequence. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. For example, a substitution mutation here would substitute a C allele for a T allele on bovine chromosome 6. Thus, a genetically modified bovine comprising a substitution mutation of the chromosomal sequence for OPN may result in bovine producing increased quantities of milk.

Milk bioactive proteins and peptides are potential health-enhancing nutraceuticals for food. Many bioactive peptides/proteins may be used as nutraceuticals, for example, in the treatment of cancer, asthma, diarrhea, hypertension, thrombosis, dental diseases, as well as mineral malabsorption, and immunodeficiency. In an exemplary embodiment, the genetically modified bovine may be a “humanized” bovine comprising at least one chromosomally integrated sequence encoding a functional human protein or peptide. The functional human protein or peptide may or may not have corresponding ortholog in the genetically modified bovine. Genetically humanized bovine will generally either express exogenous human protein or peptide, or overexpress an existing protein or peptide.

An exemplary and non-limiting group of proteins or peptides that are targeted in a genetically modified bovine includes Lactoferrin (Lf), Casein, Proline rich polypeptide (PRP), alpha-Lactalbumin (LA), Lactoperoxidase, Lysozyme. Lf is a potent inhibitor for several enveloped and naked viruses, such as rotavirus, enterovirus and adenovirus. Lf is resistant to tryptic digestion and breast-fed infants excrete high levels of faecal Lf, so that its effect on viruses replicating in the gastrointestinal tract is of great interest. Bovine with genetically modified Lf gene generally produces milk with antibacterial, antifungal, antiviral, antiparasite, antitumor, and enhanced immunomodulatory properties.

Casein has been protective in experimental bacteremia by eliciting myelopoiesis. Casein hydrolyzates were also protective in diabetic animals, reduced the tumor growth and diminished colicky symptoms in infants. PRP revealed variety of immunotropic functions, including promotion of T-cell activation and inhibition of autoimmune disorders such as multiple sclerosis. LA demonstrates antiviral, antitumor and anti-stress properties. Lactoperoxidase shows antibacterial properties. Lysozyme is effective in treatment of periodentitis and prevention of tooth decay.

In one embodiment, the exogenously expressed or overexpressed milk component is Lf, Casein, PRP, LA, Lactoperoxidase, Lysozyme or any combination thereof. The genetically modified bovine comprising an edited chromosomal sequence encoding Lf, Casein, PRP, LA, Lactoperoxidase, and/or Lysozyme may produce milk with enhanced property in disease prevention or treatment than a bovine in which said chromosomal region(s) is not edited.

The presence of pathogenic organisms in milk continues to be a problem. It has been shown that specific antibodies can be produced in transgenic animals. It might be possible to produce antibodies in the mammary gland that are capable of preventing a mastitis infection or antibodies that aid in the prevention of human diseases. Thus, one can also envision antibodies against salmonella, lysteria or other pathogens that will produce safer milk products.

Other exemplary examples of bovine chromosomal sequences to be edited include those that code for proteins related to muscle mass and meat production. For example, visibly distinct muscular hypertrophy (mh), commonly known as double muscling, occurs with high frequency in the Belgian Blue and Piedmontese cattle breeds.

The autosomal recessive mh locus causing double-muscling condition in these cattle maps to bovine chromosome 2 within the same interval as myostatin, a member of the TGF-β superfamily of genes. Because targeted disruption of myostatin in mice results in a muscular phenotype very similar to that seen in double-muscled cattle, this gene is as a candidate gene for double-muscling condition by cloning the bovine myostatin cDNA

The expression pattern and sequence of the gene in normal and double-muscled cattle has also been analyzed. The analysis demonstrates that the levels and timing of expression do not appear to differ between Belgian Blue and normal animals, as both classes show expression initiating during fetal development and being maintained in adult muscle. Moreover, sequence analysis reveals mutations in heavy-muscled cattle of both breeds. Belgian Blue cattle are homozygous for an 11-bp deletion in the coding region that is not detected in cDNA of any normal animals.

This deletion results in a frame-shift mutation that removes the portion of the Myostatin protein that is most highly conserved among TGF-β family members and that is the portion targeted for disruption in the mouse study. Piedmontese animals tested have a G-A transition in the same region that changes a cysteine residue to a tyrosine. This mutation alters one of the residues that are hallmarks of the TGF-β family and are highly conserved during evolution and among members of the gene family. It therefore appears likely that the mh allele in these breeds involves mutation within the myostatin gene and that myostatin is a negative regulator of muscle growth in cattle as well as mice. The sequence data for bovine myostatin has been submitted to GenBank under accession no. AF019761.

Cattle with two mutant genes are more strongly affected than those carrying only one affected gene. Double muscling is usually considered a disease because these cattle often have serious problems, such as difficulty giving birth. During natural birth, the mother is often injured by the muscular calf she is trying to deliver; there is also a much higher chance of injury or death to the calf. Some cattlemen have been successful raising double muscled cattle, but it requires extra work and many calves have to be delivered by c-section. Editing the chromosomal sequence to change the double mutant allele would reduce double muscling and eliminate the need for c-sections in this cattle breed.

It is generally known that Mad Cow disease (Bovine spongiform encephalopathy or BSE) in cattle is caused by the ingestion of prion proteins in BSE infected cattle. It is also known that pathogenic prions can arise spontaneously and that they cannot be destroyed by high heat. Breeding and producing cattle that are resistant to BSE or have a knocked out Prion Protein Gene (PRPN) could be an important strategy for eradicating Mad Cow disease and thereby ensuring the safety of our beef products. One study noted that the promoter region of the PRPN gene influences the expression level of the prion protein and thus the incubation period of BSE. This suggests that animals with resistance to BSE would be less likely to contract Mad Cow disease.

The immediate benefit to breeders, producers and ultimately the consumers is that selective cattle breeding programs will lead to individual animals as well as entire cattle populations with high levels of resistance to Mad Cow disease. An additional important benefit may be improving beef exports to countries currently not accepting U.S. beef with the knowledge that exported animals have resistance to Mad Cow disease and therefore are not likely to have the disease.

In a further embodiment, the genetically modified bovine may comprise an edited chromosomal sequence encoding PRPN, wherein the chromosomal sequence is inactivated such that certain alleles of PRPN protein are not produced. Furthermore, the genetically modified bovine having the inactivated PRPN chromosomal sequence described herein may exhibit reduced susceptibility to BSE. In a non-limiting embodiment, the genetically modified bovine may comprise an edited chromosomal sequence encoding PRPN. In another non-limiting embodiment, the genetically modified bovine may comprise an edited chromosomal sequence inactivating PRPN. Deletion of PRPN may also affect the horns in bulls, as microsatellites TGLA49 and BM6438 show complete linkage to the horns locus.

The main determinant of coat color in mammals is the amount and type of melanin pigment in skin and hair. Melanocytes can produce two types of pigment, eumelanin (black/brown) and phaeomelanin (yellow/red), but usually only one pigment type at a time. Binding of α-melanocyte-stimulating hormone (α-MSH) to MSH receptor (MC1-R or MC1R) on melanocyte cell surfaces initiates production of eumelanin. Absence of the α-MSH signal results in phaeomelanin production. Any number of possible alterations to the normal functioning of this complex system will result in hair, without pigmentation or a dilution of pigmentation. For instance, white color can be caused by several reasons such as the lack of melanocytes or decreased effectiveness of melanin production. Another example is the dominant dark coat color in which a mutation in MC1-R causes activation of this receptor even in the absence of α-MSH. Overexpression of MC1R is also associated with obesity. Increased obesity in cattle may be a desirable characteristic in beef production and increase the quality of the beef.

Therefore, in a further embodiment, the genetically modified bovine may comprise an edited chromosomal sequence encoding for overexpression of MC1R protein, wherein the edited chromosomal sequence comprises a mutation such that MC1R is produced in large quantities. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation causes the overproduction of MC1R when compared with bovine with an unedited chromosomal sequence. Thus, a genetically modified bovine comprising a modified chromosomal sequence may produce better quality and higher quantities of beef.

TABLE 2
Color Loci Shown to Affect Coat Color in Cattle

| Locus Name | Gene | Action | Cattle |
|---|---|---|---|
| A agouti | ASIP | shading | 13 |
| B brown | TYRP1 | dun brown in Dexter | 8 |
| C albino | TYR | albinisim in Braunvieh | 29 |
| E extension | MC1R | red versus black | 18 |
| R roan/steel | KITLG/MGF | roan in cattle | 5 |
| | KIT | spotting | 6q23 |

Referring to Table 2, red and black are the two most common coat colors in cattle. The gene causing red/black is the Melanocortin 1 Receptor gene (MC1R), formerly called the Melanocyte Stimulating Hormone Receptor Gene (MSHr). This gene has two common alleles E^{D} and e. In addition, a less common allele, E⁺, also called “wild type” occurs. When E^{D} is present in an animal, it is typically black. This is the dominant allele in the series. Cattle that are e/e are red (recessive).

However E⁺ appears to act as a “neutral” allele in most breeds and we think E^{D}/E⁺ cattle are typically black and E⁺/e cattle are typically red. E⁺/E⁺ cattle can be almost any color since other genes, such as the Agouti gene, take over in dictating what pigments are produced. Additionally, a mutation causing brindle in the Agouti gene of cattle. All brindle cattle have at least one E^{+/} allele and none have an E^{D} allele. The E^{D} allele has been reported to be nonresponsive to agouti and constitutively expressed in cattle. It is therefore probably a preferable allele in cattle which are meant to be solid black in coat color. The E⁺ allele is responsive to agouti and is the allele in cattle with shaded body color such as Brown Swiss or Braunvieh, Jersey and several other rarer European breeds.

White, as in the Charolais, is actually caused by an epistatic or masking gene. When this Charolais allele occurs in homozygous form, the animal is white. When it is only heterozygous, as in a Black Aberdeen Angus X Charolais calf the color is closer to grey or “smoke”. This white is inherited as an autosomal dominant.

Heterozygotes are not dilutions of another color but just as white as homozygotes. The white body color with colored points is caused by the tyrosinase gene. Similar patterns exist in the mouse, Himalyan rabbit and the Siamese cat and also are caused by mutations at tyrosinase.

Yellow or Pale Brown “mouse” coat colors are believed to be caused by a Diluter gene (D) or genes. D is a diluter gene where DD=dark, Dd=medium color, and dd=pale color. It appears that there is more than one diluter gene. One gene dilutes only the phaeomelanin pigments which cause red to yellow and another gene dilutes the eumelanin pigments which cause black to brown. There could be a third gene which dilutes both phaeomelanin and eumelanin and is common in Charolais cattle.

The dun colors range from brown to yellow and are inherited as a recessive trait. This color is due to a different gene than the color called dun in Galloway cattle or Highland cattle, and is mutation in the TYRP1 gene. Roan is a pattern common in Shorthorn cattle and Belgian Blue Cattle wherein the animal had intermingled colored and white hairs in at least part of its body. The pattern is inherited as the heterozygous genotype. The two homozygotes are white or colored. A relatively small proportion of the white females are sterile due to a phenomenon known as White Heifer Disease.

The gene causing the roan/white/colored phenotypes is the Mast Cell Growth Factor (MGF), also called the KIT ligand (KITLG) on cattle chromosome 5. A single base pair change in this gene is the causative mutation. Recently the amount of spotting on Holsteins has also been attributed to a gene a cattle chromosome 6 near KIT (the same gene as in Hereford whiteface). This random spotting, not including the face is thought to be inherited as a recessive trait.

Whiteface is a pattern where most or part of the face is white against a differently colored body. A recent study suggests the gene causing whiteface in Herefords is the KIT oncogne on cattle chromosome 6. A potential side effect of whiteface is a greater susceptibility to Cancer Eye or Bovine Squamous Cell Carcinoma. This is a rapidly progressing cancer which often begins with the nictating membrane or third eyelid.

Brindle is a pattern of intermingled colors which are more marbled or streaked than roan and it has been concluded the interaction of 2 genes is needed. It appears that cattle must have an E⁺ allele at the MC1R gene. Cattle with an “e/e” genotype can carry brindle but not show this phenotype. Brindle has been reported to be caused by an allele of the agouti signal peptide (ASIP). —the A^{br} allele; however, brindle cattle always have at least one E⁺ allele and no E^{D} allele.

Evidence also supports the existence of a dominant gene (designated as the slick hair gene) that is responsible for producing a very short, sleek hair coat. Cattle with slick hair were observed to maintain lower rectal temperatures (RT). The gene is found in Senepol cattle and criollo (Spanish origin) breeds in Central and South America. This gene is also found in a Venezuelan composite breed, the Carora, formed from the Brown Swiss and a Venezuelan criollo breed.

Data from Carora×Holstein crossbred cows in Venezuela also support the concept of a major gene that is responsible for the slick hair coat of the Carora breed. Cows that were 75% Holstein:25% Carora in breed composition segregated with a ratio that did not differ from 1:1, as would be expected from a backcross mating involving a dominant gene. The effect of the slick hair gene on RT depended on the degree of heat stress and appeared to be affected by age and/or lactation status.

The decreased RT observed for slick-haired crossbred calves compared to normal-haired contemporaries ranged from 0.18 to 0.4° C. An even larger decrease in RT was observed in lactating Carora×Holstein F₁ crossbred cows, even though it did not appear that these cows were under severe heat stress.

Additionally, it appears slick-haired calves grow faster immediately following weaning and that their growth during the cooler months of the year was not compromised significantly by their reduced quantity of hair. In the Carora×Holstein crossbred cows there is also a positive effect of slick hair on milk yield under dry, tropical conditions. Chromosomal editing that includes modification of the normal hair gene to the slick hair allele has numerous potential for bovine that better withstand summer temperatures and exhibit faster growth—advantageous for meat production.

Bulldog dwarfism in Dexter cattle is one of the earliest single-locus disorders described in animals. Affected fetuses display extreme disproportionate dwarfism, reflecting abnormal cartilage development (chondrodysplasia). Typically, they die around the seventh month of gestation, precipitating a natural abortion.

Heterozygotes show a milder form of dwarfism, most noticeably having shorter legs. Homozygosity mapping in candidate regions in a small Dexter pedigree suggested aggrecan (ACAN) as the most likely candidate gene. Mutation screening revealed a 4-bp insertion in exon 11 (2266_{—}2267insGGCA) (called BD1 for diagnostic testing) and a second, rarer transition in exon 1 (−198 C>T) (called BD2) that cosegregate with the disorder. In chondrocytes from cattle heterozygous for the insertion, mutant mRNA is subject to nonsense-mediated decay, showing only 8% of normal expression. Genotyping in Dexter families throughout the world shows a one-to-one correspondence between genotype and phenotype at this locus.

Mannosidosis, an inherited and lethal lysosomal storage disease of Aberdeen Angus cattle, was diagnosed on a farm in north-east Scotland. Two affected calves were examined in detail. Both were poorly grown and ataxic, though the intention tremor and aggression considered characteristic of the disease were not recorded. Histological examination revealed typical vacuolation of nerve cells, fixed macrophages and epithelial cells of the viscera. Deficiency of the enzyme alpha mannosidase has been identified as the proximal cause of the disease.

Therefore, in yet another embodiment, the genetically modified bovine may comprise an edited chromosomal sequence encoding for slick hair gene, ACAN or alpha mannosidase, wherein the edited chromosomal sequence comprises a mutation. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. For example, the nonsense, deletion, or insertion mutation results in the increased production of alpha mannosidase of when compared with bovine diagnosed with mannosidosis with an unedited chromosomal sequence. Thus, a genetically modified bovine comprising a modified chromosomal sequence may not exhibit the phenotypic characteristics of mannosidosis.

The present disclosure also encompasses a genetically modified bovine comprising any combination of the above described chromosomal alterations. For example, the genetically modified bovine may comprise an inactivated alpha-lactalbumin gene and/or edited PRPN chromosomal sequence, a modified slick hair chromosomal sequence, and/or a modified or inactivated acetyl CoA carboxylase chromosomal sequence.

Additionally, the bovine disease- or trait-related gene may be modified to include a tag or reporter gene or genes as are well-known. Reporter genes include those encoding selectable markers such as cloramphenicol acetyltransferase (CAT) and neomycin phosphotransferase (neo), and those encoding a fluorescent protein such as green fuorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

The genetically modified bovine may be heterozygous for the edited chromosomal sequence or sequences. In other embodiments, the genetically modified bovine may be homozygous for the edited chromosomal sequence or sequences.

The genetically modified bovine may be a member of one of the known bovine breeds. As used herein, the term “bovine” encompasses embryos, fetuses, newborns, juveniles, and adult bovine organisms. In each of the foregoing iterations of suitable bovines for the invention, the bovine does not include exogenously introduced, randomly integrated transposon sequences.

### (II) Genetically Modified Bovine Cells

A further aspect of the present disclosure provides genetically modified bovine cells or cell lines comprising at least one edited chromosomal sequence. The disclosure also encompasses a lysate of said cells or cell lines. The genetically modified bovine cell (or cell line) may be derived from any of the genetically modified bovines disclosed herein. Alternatively, the chromosomal sequence may be edited in a bovine cell as detailed below.

The bovine cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The bovine cell or cell line may be derived from lung (e.g., AKD cell line), kidney (e.g., CRFK cell line), liver, thyroid, fibroblasts, epithelial cells, myoblasts, lymphoblasts, macrophages, tumor cells, and so forth. Additionally, the bovine cell or cell line may be an bovine stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Similar to the genetically modified bovines, the genetically modified bovine cells may be heterozygous or homozygous for the edited chromosomal sequence or sequences.

### (III) Zinc Finger-Mediated Genome Editing

In general, the genetically modified bovine or bovine cell, as detailed above in sections (I) and (II), respectively, is generated using a zinc finger nuclease-mediated genomic editing process. The process for editing a bovine chromosomal sequence comprises: (a) introducing into a bovine embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. The embryo used in the above described method typically is a fertilized one-cell stage embryo.

Components of the zinc finger nuclease-mediated method of genome editing are described in more detail below.

### (a) Zinc Finger Nuclease

The method comprises, in part, introducing into an bovine embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fok I cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:I538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the bovine or it may be an exogenous sequence. Additionally, the sequence to be integrated may be operably linked to an appropriate control sequence or sequences. The size of the sequence to be integrated can and will vary. In general, the sequence to be integrated may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the bovine embryo or cell. Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo or Cell

The method for editing a chromosomal sequence using a zinc finger nuclease-mediated process further comprises culturing the embryo or cell comprising the introduced nucleic acid(s) to allow expression of the zinc finger nuclease.

An embryo may be cultured in vitro (e.g., in cell culture). Typically, the bovine embryo is cultured for a short period of time at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the bovine species. Routine optimization may be used, in all cases, to determine the best culture conditions for a particular species of embryo. In some cases, a cell line may be derived from an in vitro-cultured embryo (e.g., an embryonic stem cell line).

Preferably, the bovine embryo will be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair pathway. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is modified.

The genetically modified bovines disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified bovines disclosed herein may be crossed with other bovines to combine the edited chromosomal sequence with other genetic backgrounds. By way of non-limiting example, suitable genetic backgrounds may include wild-type, natural mutations giving rise to known bovine phenotypes, targeted chromosomal integration, non-targeted integrations, etc.

### (IV) Applications

The animals and cells disclosed herein may have several applications. In one embodiment, the genetically modified bovine comprising at least one edited chromosomal sequence may exhibit a phenotype desired by humans. For example, inactivation of the chromosomal sequence encoding Agouti may result in bovine producing wool with striped color coat. In other embodiments, the bovine comprising at least one edited chromosomal sequence may be used as a model to study the genetics of coat color, coat pattern, and/or hair growth. Additionally, a bovine comprising at least one disrupted chromosomal sequence may be used as a model to study a disease or condition that affects humans or other animals. Non-limiting examples of suitable diseases or conditions include albinism, hair disorders, and baldness. Additionally, the disclosed bovine cells and lysates of said cells may be used for similar research purposes.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of PRPN in Model Organism Cells

Zinc finger nuclease (ZFN)-mediated genome editing may be tested in the cells of a model organism such as a bovine using a ZFN that binds to the chromosomal sequence of a prion protein gene of the bovine cell such PRPN. The particular gene to be edited may be a gene having identical DNA binding sites to the DNA binding sites of the corresponding bovine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into bovine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected PRPN gene locus in bovine cells using a ZFN.

### Example 2

### Genome Editing of PRPN in Model Organism Embryos

The embryos of a model organism such as a bovine may be harvested using standard procedures and injected with capped, polyadenylated mRNA encoding a ZFN similar to that described in Example 1. The bovine embryos may be at the one cell stage when microinjected. Control embryos may be injected with 0.1 mM EDTA. The frequency of ZFN-induced double strand chromosomal breaks may be estimated using the Cel-1 assay as described in Example 1. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

What is claimed is:

1. A genetically modified bovine comprising at least one edited chromosomal sequence.

2. The genetically modified bovine of claim 1, wherein the edited chromosomal sequence is inactivated, is modified, or has an integrated sequence.

3. The genetically modified bovine of claim 1, wherein the edited chromosomal sequence encodes a protein chosen from alpha s1 casein, beta casein, kappa casein, alpha s2 casein, plasmin, chymosin, bovine serum albumin, lactose, lactase, galactose, glucobeta lactaglobulin, alpha lactalbumin, lactoferrin, RIPTAC, osteopontin, acetyl coA carboxylase, diacylglyercol acyl transferase 1, and combinations thereof.

4. The genetically modified bovine of claim 3, wherein the protein is plasmin, and the edited chromosomal sequence comprises at least one mutation such that the sequence is inactivated and the protein is not produced or the protein is not functional.

5. The genetically modified bovine of claim 4, wherein the bovine produces milk without plasmin-cleaved casein proteins.

6. The genetically modified bovine of claim 3, wherein the protein is osteopontin, and the edited chromosomal sequence comprises at least one mutation such that the sequence is modified and the expressed protein comprises at least one amino acid change.

7. The genetically modified bovine of claim 6, wherein the bovine exhibits increased milk production compared with a bovine in which the chromosomal region is not edited.

8. The genetically modified bovine of claim 1, wherein the protein is PRPN, TGF-β, BST, BGH, MC1R, or ACAN, and combinations thereof and the edited chromosomal sequence comprises at least one mutation such that the sequence is modified.

9. The genetically modified bovine of claim 8, wherein the protein is ACAN and the edited chromosomal sequence comprises at least one mutation such that the sequence is modified or inactivated.

10. The genetically modified bovine of claim 9, wherein the bovine in which the chromosomal region is edited does not exhibit a bulldog dwarfism phenotype.

11. The genetically modified bovine of claim 3, wherein the protein is PRPN, and the edited chromosomal sequence comprises at least one mutation such that the sequence is modified and the expressed protein comprises at least one amino acid change.

12. The genetically modified bovine of claim 11, wherein the bovine has a different susceptibility to BSE than a bovine in which the chromosomal sequence is not edited.

13. The genetically modified bovine of claim 3, wherein the protein is acetyl coA carboxylase and the edited chromosomal sequence comprises at least one mutation such that the sequence is inactivated.

14. The genetically modified bovine of claim 13, wherein the bovine produces milk with less fat content than a bovine without the genetic modification.

15. The genetically modified bovine of claim 1, wherein the bovine is heterozygous or homozygous for the edited chromosomal sequence.

16. The genetically modified bovine of claim 1, wherein the bovine is an embryo, a calf, or an adult.

17. A bovine embryo, the embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

18. The bovine embryo of claim 17, wherein the chromosomal sequence encodes a protein chosen from alpha s1 casein, beta casein, kappa casein, alpha s2 casein, plasmin, chymosin, bovine serum albumin, lactose, lactase, galactose, glucobeta lactaglobulin, alpha lactalbumin, lactoferrin, RIPTAC, osteopontin, acetyl coA carboxylase, diacylglyercol acyl transferase 1 and combinations thereof.

19. A genetically modified bovine cell comprising at least one edited chromosomal sequence.

20. The genetically modified bovine cell of claim 19, wherein the edited chromosomal sequence encodes a protein chosen from alpha s1 casein, beta casein, kappa casein, alpha s2 casein, plasmin, chymosin, bovine serum albumin, lactose, lactase, galactose, glucobeta lactaglobulin, alpha lactalbumin, lactoferrin, RIPTAC, osteopontin, acetyl coA carboxylase, diacylglyercol acyl transferase 1 and combinations thereof.
